(19) United States
(12) Patent Application Publication
(10) Pub. No.: US 2013/0102055 A1
(43) Pub. Date: Apr. 25, 2013

(54) CONTINUOUS FLOCCULATION DEFLOCCULATION PROCESS FOR EFFICIENT HARVESTING OF MICROALGAE FROM AQUEOUS SOLUTIONS

(71) Applicant: BOARD OF REGENTS, THE UNIVERSITY OF TE, Austin, TX (US)

(72) Inventors: Lynn E. Katz, Austin, TX (US); Kerry A. Kinney, Austin, TX (US); Jinyong Choi, Austin, TX (US); Eric Chen, Austin, TX (US)

(73) Assignee: BOARD OF REGENTS, THE UNIVERSITY OF TEXAS SYSTEM, Austin, TX (US)

(21) Appl. No.: 13/656,691

(22) Filed: Oct. 20, 2012

Related U.S. Application Data
(60) Provisional application No. 61/549,579, filed on Oct. 20, 2011.

(51) Int. Cl. C12N 1/12; C12N 1/20; C12M 1/42; C12N 7/02

(52) U.S. Cl. 435/239; 435/252.1; 435/257.1; 435/257.3; 435/257.4; 435/257.5; 435/257.6; 435/292.1

(57) ABSTRACT

A continuous process for efficiently harvesting microalgae from aqueous systems is described herein. The method and apparatus of the present invention allows continuous harvesting of algae from a variety of source waters including saltwater, brackish water, fresh water, and treated wastewater. High concentration factors are achievable and the system produces a deflocculated product that is readily processed for biofuel or pharmaceutical applications. The process of the present invention does not add contaminants that can limit the downstream usage possibilities for the algae concentrate produced. The effluent water from the process is suitable for conventional discharge or recycling to the growth system. The process of the present invention is inexpensive, scalable, and generates useful effluent water and algae concentrate as products.

### CROSS-REFERENCE TO RELATED APPLICATIONS

This application claims priority to U.S. Provisional Application Ser. No. 61/549,579, filed Oct. 20, 2011, the entire contents of which are incorporated herein by reference.

### TECHNICAL FIELD OF THE INVENTION

The present invention relates in general to producing commercially valuable products from algae, such as biofuels, pharmaceuticals, nutraceuticals, and lipids by processing biomass and wastewater, and more particularly, to a process for producing a deflocculated, pumpable microalgal concentrate from dilute aqueous solutions.

### STATEMENT OF FEDERALLY FUNDED RESEARCH

None.

### REFERENCE OF A SEQUENCE LISTING

None.

### BACKGROUND OF THE INVENTION

Without limiting the scope of the invention, its background is described in connection with microalgal harvesting systems and processes.

U.S. Patent Application Publication No. 2011/0081706 (Schlesinger et al. 2011) provides a technique for harvesting algae using Ca(OH)₂ and/or Mg(OH)₂ for which the doses are dependent on the logarithm of cell density. The process has been applied in batch mode to saltwater species. The supernatant from the process can be recycled back to the culture using a standard recarbonation process with addition of phosphoric acid presumably for the growth media. The goal of the invention is to produce a flocculated algae paste or mat to couple with centrifugation, filtration or dissolved air flotation.

U.S. Patent Publication Application No. 2010/0144017 (Shepherd, 2010) provides a system for harvesting algae in continuous fermentation. There is a harvester including a main moving belt, a plurality of rollers, and a motor for driving the main moving belt. There is a reactor tank and a vacuum extractor for applying a vacuum over a width of the main moving belt to extract biomass and to dry the main moving belt. The main moving belt has one end in the reactor tanks and another end extended into the vacuum extractor. The algae contained in the reactor tank is collected for further processing, including oil extraction. With algae harvested in the large-scale manner of the present invention, a more efficient oil extraction method can be used because the concentration, temperature, and pressure can be more easily controlled.

U.S. Pat. No. 6,524,486 (Borodyanski and Konstantinov, 2003) relates to an apparatus and method for separating microalgae from water without rupturing cells. The method described in the '486 patent comprises the steps of flocculation, flotation and dehydration. Microalgae suspension from a reservoir is passed to a mixer unit where flocculation is carried out, using modified starch or other flocculating agents. The suspension is then directed to a flotation column. Dissolved gas in water is transferred to the flotation column through a disperser. A layer of foam containing microalgae is formed on the liquid layer in the column, which can be skimmed off through an overflow outlet. The flotation column is a telescopic column of adjustable height, which enables the position of the overflow outlet to be aligned with the level of the foam layer for efficient foam removal. Foam containing microalgae is then passed to a filtration unit for cloth filtration, followed by drying in a drying chamber.

### SUMMARY OF THE INVENTION

The present invention describes a process for producing a deflocculated concentrate from dilute aqueous systems containing one or more biological cells, non-limiting examples of biological cells include microalgae, cyanobacteria, pathogenic organisms and biosolids. The process is applicable to saltwater, brackish water, fresh water, and treated wastewater or water recovered from wastewater solids, and removal is independent of initial organism concentration within dilute aqueous streams. The process described herein results in high concentration factors and produces a deflocculated product that is useful itself and readily compatible with subsequent processing methods to yield commercially valuable products. Non-limiting applications wherein the process of the present invention can be used include algal biomass production for: biofuel production, pharmaceuticals and nutraceutical manufacture, wastewater treatment, lipid production, etc. The algae concentrate produced in the process of the present invention is free of external contaminants that can limit the downstream usage possibilities for the concentrate. The process of the present invention produces a homogeneous microalgae slurry that is pumpable and suitable for downstream extraction in a membrane extraction system as well as other more traditional lipid and oil extraction systems. The process of the present invention is applicable to all types of algae and it produces a homogenous, pumpable concentrate. Furthermore, the process does not necessarily entail addition of any organics or heavy metals to the water, leading to protection of water quality and greater use of the algal product that is obtained by the process described herein. The water effluent from the process can be discharged or recycled to the algae growth system using traditional recarbonation processes thereby allowing the large volumes of water are required to grow algae to be recycled. The process of the present invention can be used in algal biomass production for: biofuel production, pharmaceuticals, food supplements, tertiary treatment of municipal wastewater, lipid production, and metal extraction.

The present invention in one embodiment provides a method for harvesting or separating one or more biological cells from an aqueous feed or stream comprising the steps of: (i) providing the aqueous feed or stream comprising the one or more biological cells in a tank or a vessel; (ii) precipitating one or more solids in the aqueous feed or stream, wherein the one or more biological cells are reversably attached to the precipitated solids; (iii) allowing the precipitated solids to settle to a bottom portion of the tank or the vessel; (iv) separating an aqueous supernatant from the settled solid precipitate; and (v) acidifying the settled solid precipitate, wherein the acidification results in a separation or a release of one or more biological cells from the solid precipitate to form a concentrated slurry comprising the one or more biological cells.

In one aspect of the method described hereinabove a pH of the aqueous feed or stream may be modified prior to the precipitation of the one or more solids, wherein the modification results in an increase or a decrease in the pH. In a related aspect the pH of the aqueous feed or stream is modified by the addition of one or more bases, chemicals, or metals selected from the group consisting of lime, NaOH, KOH, NH₄OH, Ca(OH)₂, Mg(OH)₂, alum, aluminium chlorohydrate, aluminium sulfate, calcium oxide, iron(II) sulfate, iron(III) chloride, polyacrylamide, polyDADMAC, sodium aluminate, sodium silicate, chitosan, guar gum, alginates, and gelatin.

In another aspect the method is operated as an independent standalone operation or is incorporated or is in communication with an algal processing platform or unit. In yet another aspect the method further comprises the optional steps of:
a) adjusting the pH of the separated aqueous supernatant, wherein the pH adjusted supernatant is discharged as an effluent or is recycled to a grow one or more biological cells; and
b) processing the concentrated slurry comprising the one or more biological cells in a processing unit to yield an oil or biodiesel, a biofuel, a pharmaceutical product, a nutraceutical product, a lipid product, or any combinations thereof, wherein the processing unit comprises:
(i) one or more lysing units to electromechanically lyse the one or more biological cells by an application of an electromagnetic field, wherein the lysis results in a release of one or more cellular components comprising oils, neutral lipids, proteins, triglycerides, sugars or combinations and modifications thereof from the biological cells;
(ii) one or more separations unit to separate the released oils and lipids from the medium resulting in a generation of a residual biomass; and
(iii) one or more optional pumping equipment, heat exchangers, distilling equipment, reboilers, condensors, and combinations and modifications thereof.

In one aspect processing unit may optionally comprise:
(i) one or more conversion units to convert the oils, neutral lipids or triglycerides to a fatty acid methyl ester (FAME), a biodiesel, a biofuel, a pharmaceutical product, a nutraceutical product, a lipid product, or any combinations thereof;
(ii) one or more processing units to process the residual biomass for disposal, for conversion to methane or other chemicals, for conversion to animal feed, or any combinations thereof; and
(iii) one or more storage tanks, vessels or containers to store released and separated cellular components or the processed biodiesel or biofuel.

In another aspect the step of processing the concentrated slurry comprising the one or more biological cells in a processing unit to yield an oil or biodiesel, a biofuel, a pharmaceutical product, a nutraceutical product, a lipid product, or any combinations thereof comprises the steps of:
(i) pumping or transferring the concentrated aqueous slurry comprising the one or more biological cells to the one or more lysing units;
(ii) lysing the one or more cells electromechanically to release one or more cellular components comprising oils, neutral lipids, proteins, triglycerides, sugars or combinations and modifications thereof from the biological cells; and
(iii) separating the released oils and lipids from the medium in the separations units resulting in a generation of a residual biomass.

Method and techniques for electromechanical lysing to release one or more cellular components have been previously described by the present inventors in U.S. patent application Ser. Nos. 12/198,443 and 13/186,282 and in U.S. Patent Application Publication No. 2009/0087900 (relevant portions incorporated herein by reference). It will be understood by a skilled artisan that a number of techniques may be employed for separation of the released oils and lipids. The present inventors have previously described a microporous membrane based method for recovering oil from a lysed algal concentrate in U.S. patent application Ser. No. 13/006,342, entitled “Non-Dispersive Process for Insoluble Oil Recovery From Aqueous Slurries”, relevant portions of which are incorporated herein by reference.

In yet another aspect the method of the present invention further comprising the steps of:
(i) converting the oils, neutral lipids or triglycerides to a fatty acid methyl ester (FAME), a biodiesel or a biofuel, a pharmaceutical product, a nutraceutical product, a lipid product, or any combinations thereof; and
(ii) processing the residual biomass for disposal, for conversion to methane or other chemicals, for conversion to animal feed, or any combinations thereof.

In one aspect of the method the processing unit may be a stationary processing plant or a modular mobile unit on a transportable platform. In a specific aspect the platform is a trailer bed or a trailer. In another aspect the platform comprises one or more sets of wheels to enable fastening onto a transportation unit. In yet another aspect the modular mobile unit comprises: one or more power supply units to provide electricity to run the lysing and separations units and to remotely operate the unit and one or more control panels to operate and monitor the performance of the lysing and separations units.

In one aspect the one or more biological cells comprise algal cells, bacterial cells, viral cells or combinations thereof. In another aspect one or more algal cells comprise microalgae selected from a class comprising Bacillariophyceae, Eustigmatophyceae, and Chrysophyceae. In yet another aspect the microalgal genera are selected from the group consisting of Nannochloropsis, Chlorella, Dunaliella, Scenedesmus, Selenastrum, Oscillatoria, Phormidium, Spirulina, Amphora, and Ochromonas. In a related aspect the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

In one aspect the aqueous feed or stream comprises saltwater, brackish water, fresh water, treated wastewater or combinations thereof. In another aspect the acidification of the solid precipitate is achieved by addition of one or more acids or CO₂. In yet another aspect the present invention describes one or more biological cells from an aqueous feed or stream harvested by the method of the present invention.

Another embodiment of the present invention relates to a method for harvesting or separating one or more microalgal cells from an aqueous feed or stream comprising the steps of: i) providing the aqueous feed or stream comprising the one or more microalgal cells in a tank or a vessel, wherein the aqueous feed or stream comprises saltwater, brackish water, fresh water, treated wastewater or combinations thereof; ii) raising a pH of the aqueous feed or stream by an addition of a base or lime; iii) precipitating one or more solids in the aqueous feed or stream, wherein the one or more microalgal cells are associated with the precipitated solids; iv) allowing the precipitated solids to settle to a bottom portion of the tank or the vessel; v) separating an aqueous supernatant from the settled solid precipitate; and vi) contacting the settled solid precipitate with CO₂ or other acid to acidify the solid precipitate, wherein the acidification results in a separation or a release of the one or more microalgal cells from the solid precipitate to form a concentrated slurry comprising the one or more microalgal cells. In one aspect the present invention discloses one or more microalgal cells harvested by the method hereinabove.

In yet another embodiment the present invention discloses an apparatus for producing a biodiesel, a fatty acid methyl ester (FAME), a biofuel or combinations and modifications thereof from a microalgal cell culture comprising:
i) an algal growth tank or a cultivation tank comprising an aqueous feed or stream for growing the one or more algal species in presence of water and other growth factors selected from the group consisting of nutrients, minerals, CO₂, air, and light;
ii) a harvesting tank for separating or harvesting the microalgal cell culture from the aqueous feed or stream, wherein the aqueous feed or stream comprises saltwater, brackish water, fresh water, treated wastewater or combinations thereof, wherein the method of harvesting or separating the microalgal cell culture comprises the steps of:
   a) raising a pH of the aqueous feed or stream by an addition of a base;
   b) precipitating one or more solids in the aqueous feed or stream, wherein the microalgal cells are associated with the precipitated solids;
   c) allowing the precipitated solids to settle to a bottom portion of the tank or the vessel;
   d) separating an aqueous supernatant from the settled solid precipitate; and
   e) contacting the settled solid precipitate with CO₂ or other acid to acidify the solid precipitate, wherein the acidification results in a separation or a release of the microalgal cells from the solid precipitate to form a concentrated slurry comprising the one or more microalgal cells; and
iii) a processing unit for processing the concentrated slurry of the microalgal cells comprising:
a) one or more lysing units to electromechanically lyse the one or more microalgal cell culture by an application of an electromagnetic field, wherein the lysis results in a release of one or more cellular components comprising oils, neutral lipids, proteins, triglycerides, sugars or combinations and modifications thereof from the algal cells;
b) one or more separations unit to separate the released oils and lipids from the medium resulting in a generation of a residual biomass;
c) a reaction vessel for converting the separated algal lipids, triglycerides to a biodiesel, a FAME, a biofuel or combinations or modifications thereof by a transesterification reaction; and
d) one or more optional pumping equipment, heat exchangers, distilling equipment, reboilers, condensors, and combinations and modifications thereof;
e) one or more power supply units to provide electricity to run the dewatering, lysing, and separations units and to remotely operate the unit; and
f) one or more control panels to operate and monitor the performance of the dewatering, lysing, and separations units.

In one aspect the apparatus as disclosed hereinabove is capable of operation in a batch or a continuous processing mode. In another aspect the apparatus is operated to recirculate some of the flocculated solids back into the incoming dilute algal stream to promote faster and more efficient flocculation. In yet another aspect one or more lysing units comprise electromechanical lysing units, sonicators, ultrasound devices, pressure homogenizers, high speed homogenizers, osmotic shock inducing devices or devices for chemical or enzymatic lysis. In yet another aspect the separation units comprise non-dispersive separation devices, decantation units, liquid-liquid extraction units, solvent assisted extraction units or combinations and modifications thereof. In a specific aspect the microalgal culture is Chlorella or Nannochloropsis.

### BRIEF DESCRIPTION OF THE DRAWINGS

For a more complete understanding of the features and advantages of the present invention, reference is now made to the detailed description of the invention along with the accompanying figures and in which:

FIG. 1 is a general process flow diagram showing the steps in the continuous flocculation deflocculation process for the concentration of microalgae according to an embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein the term “algae” represents a large, heterogeneous group of primitive organisms which occur throughout all types of aquatic habitats and moist terrestrial environments. Nadakavukaren et al., Botany. An Introduction to Plant Biology, 324-325, (1985). The term “algae” as described herein is intended to include the species selected from the group consisting of the diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nanochloropsis, Tetraselmis, Chlorella, Dunaliella, Oscillatoria, Synechococcus, Boekelovia, Isochysis and Pleurochysis.

The algal cells described hereinabove are selected from a division comprising Chlorophyta, Cyanophyta (Cyanobacteria), Rhodophyta (red algae), and Heterokontophyt. The one or more algal cells comprise microalgae selected from a class comprising Bacillariophyceae, Eustigmatophyceae, and Chrysophyceae. The microalgal genera are selected from the group consisting of Nannochloropsis, Chlorella, Dunaliella, Scenedesmus, Selenastrum, Oscillatoria, Phormidium, Spirulina, Amphora, and Ochromonas. In yet another aspect the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

The instant invention describes a process to produce a deflocculated algae or biomass concentrate from dilute aqueous solutions. The biomass resulting from the process of the present invention may be processed into a liquid biofuel or into other products that can utilize the biomass including animal feed, biogas (methane generation) or platform chemical production.

The invention described herein comprises two major processes in series (flocculation of the algae to remove it from the feed water followed by deflocculation to separate the algae from the precipitated solids). The continuous-feed flocculation process is achieved by adding lime or other base (e.g., NaOH) to the feed solution to rapidly raise the pH of the aqueous solution. The addition of ions such as Mg or Ca may be required depending on the composition of the background water. For example, if the quality of the water stream is not conducive for optimal flocculation pretreatment may be required, e.g., if the water is hard and has a high alkalinity, the water may be pre-treated by addition of acid and air sparging, prior to the precipitation process. The rapid pH rise in the main process leads to precipitation of the inorganic constituents in the feed water and association of the microalgae with the precipitate. Release of the algae or biomass requires dissolution of the precipitate, which is facilitated through pH reduction via carbon dioxide or other acid such as HCl. In another embodiment of the invention, base addition modifies the surface charge characteristics of microalgae and causes the biomass to flocculate with minimal formation of inorganic precipitate. In this scenario, low Mg and Ca concentrations are required in the water. In either case, the flocculated algae or the flocculated algae enmeshed in the inorganic precipitate settles rapidly to the bottom of a continuous flow plate or tube settler. The microalgae is thus removed from the feed solution. In certain operating modes, a stream of flocculated algae will be recirculated into the feed tank to promote faster and more efficient flocculation of dilute algae. The treated effluent water is suitable (after pH adjustment) for discharge and potentially for recycle to the growth pond. The biomass enmeshed in the inorganic precipitate (or flocculated) are deflocculated in a continuous flow deflocculation process that utilizes contact with carbon dioxide or other acid to reacidify the precipitated solids and release the microalgae or other biomass. The resulting product is a homogenous slurry of biomass that has been recovered from the feed solution. Release of the algae from the precipitated solids can be enhanced by mechanical agitation. It is also an intent of the present invention to recycle any residual precipitated solids as seed to reduce the base requirement.

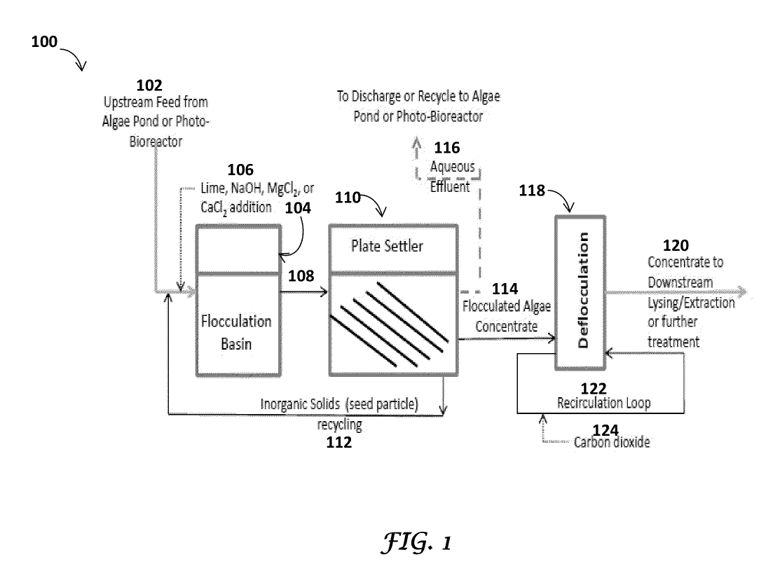

The continuous flocculation deflocculation process of the present invention is depicted in a schematic process diagram 100 as shown in FIG. 1. An upstream feed 102 from an algae pond or photo-bioreactor is fed to a flocculation basin or vessel 104. The continuous-feed flocculation process is achieved by adding lime or other base (e.g., NaOH, Mg(OH)₂) 106 to the feed solution 102 through an in-line static mixer or a separate rapid mix step (not shown) to rapidly raise the pH of the aqueous solution prior to entering the flocculation basin 104. The addition of ions such as Mg or Ca in the stream 106 may be required depending on the composition of the background water. The rapid pH rise leads to precipitation of the inorganic constituents in the feed water 102 and incorporation of the microalgae in the precipitate. Release of the algae or biomass requires dissolution of the precipitate, which is facilitated through pH reduction via carbon dioxide or other acid such as HCl or phosphoric acid. In low hardness waters, base (in stream 106) addition modifies the surface charge characteristics of microalgae and causes the biomass to flocculate with minimal formation of inorganic precipitate. In either the enmeshment or flocculation scenarios, the flocculated algae or the flocculated algae enmeshed in the inorganic precipitate 108 settles rapidly to the bottom of a continuous flow plate or tube settler 110. The microalgae is thus removed from the feed solution 102 and the treated effluent water 116 is suitable (after pH adjustment in some cases) for discharge and potentially for recycle to the growth pond or photobioreactor, or for reuse for other applications. The biomass associated with the inorganic precipitate (or flocculated) 114 are deflocculated in a continuous flow deflocculation process 118 that utilizes contact with carbon dioxide 124 to dissolve the precipitated solids and release the microalgae or other biomass. The resulting product is a homogenous slurry of biomass 120 that has been recovered from the feed solution. Release of the algae from the precipitated solids can be enhanced by mechanical agitation or addition of additional acid. A portion of the precipitated solids 112 can be recirculated as seed to promote faster flocculation of dilute algae, thereby reducing the base requirement in stream 106.

One skilled in the art will understand that alternate possible configurations are obvious, for example CO₂ 124 may be added directly to the deflocculation tank 118 without relying on a recirculation loop 122.

Depending on the chemistry of the aqueous media in which the algae are growing, the ratio of Mg/Ca varies. The amount of base needed is tied directly to the alkalinity or acidity of the water and the ability to utilize recycled solids for pH control. In seawater, Mg concentrations are sufficient for removal strictly via lime addition and in some cases solely using recycled concentrate. In other cases, sodium hydroxide alone is used for pH adjustment especially when Ca and Mg concentrations allow for charge neutralization processes. Similarly, depending on the composition of the aqueous media, the base addition may be supplemented with the addition of magnesium chloride to enhance flocculation of the algae. Ratios of Ca/Mg are optimized empirically to minimize costs and formation of the precipitate and maximize removal of the biomass.

Inorganic precipitates that form in the first flocculation process can be recycled and blended with the algae feed water to act as nucleation sites (seed particles) to enhance flocculation/precipitation reactions and lower the pH and/or chemical dosages required to flocculate the algae in the feed water. These recycled seed particles may be recovered from the feed water prior to the deflocculation step. In certain embodiments, the pH of the aqueous feed or recycle stream may be modified prior to the precipitation of the one or more solids which may include modifying a water that is hard and has a high alkalinity by addition of acid and air sparging, prior to the precipitation process. The unique features of the technology described in the present invention include: are as follows: (i) the process yields deflocculated biomass that is not contaminated with flocculants (e.g., metals, polymers, organics) that make the harvested biomass unsuitable for many downstream applications, (ii) the process described herein is a continuous flow process in which reagents (for e.g., base and carbon dioxide) may be added to achieve high removal efficiencies, (iii) the process generates a homogeneous biomass slurry that is suitable for membrane extraction as well as more traditional lipid and oil extraction processes such as solvent extraction, (iv) the process is cost effective because the water is not necessarily contaminated with reagents, and (v) the relationship between chemical dosage and algae cell concentration is not logarithmic. As a result, the water effluent from this harvesting process is suitable for discharge or recycling back to the microalgae production pond for reuse. This greatly reduces the water consumption of microalgae production process, (v) the process as described previously is a continuous process that allows algae to be continuously harvested and deflocculated from the growth ponds/solutions if desired, and (vi) the process as described herein offers advantages over present continuous flow technologies that rely on expensive membrane separations (that are mechanically cumbersome and expensive) or the addition of expensive flocculants that greatly limit the value of the harvested algae. Also, many of the technologies under development are not suitable or economical for scaling up to processing million gallons per day of process water.

The processes and the systems of the present invention address some of the problems with existing technologies and methods. These are described herein below:
(i) Current large-scale microalgae production systems for biofuel applications generally yield dilute solutions (for e.g., <1 g/L) or highly concentrated solutions of microalgae. A harvesting process that is not logarithmically concentration dependent and can efficiently process both dilute and concentrated solutions of microalgae from a variety of source waters (e.g., saltwater, brackish water, fresh water, and treated wastewater or water recovered from wastewater solids) is critical for the viability of microalgae production for biofuel applications;
(ii) Current technologies are generally not suitable for large scale applications since they are not practical for scaling up. These technologies are also expensive for handling large volumes of water, and tend to contaminate the algae product;
(iii) The process of the present invention yields an algae slurry concentrate which is suitable for column contactors or other proprietary lipid extraction systems whereas a dry, flocculated product is not; and
(iv) The pumpable microalgae product is potentially suitable for several downstream applications including utilization in the anaerobic digestor for biogas generation, production of specialty chemicals or as a biomass source for conversion into platform chemicals.

The capabilities of the flocculation/deflocculation unit are summarized in the Table 1 herein below.

TABLE 1
Capabilities of the flocculation/deflocculation unit.

| Parameter | Operating Range |
|---|---|
| Algae Concentration in Feed Water | Minimum of 50 mg |
| | algae dry weight per L |
| Operating pH | 9.3 to 12.2 |
| Aqueous effluent pH | 6.5 to 9 |
| Algae Concentration Factor | 20X to 150X |

Other features of the system of the present invention include: (1) modular system that can be skid mounted and delivered to the algae pond or bioreactor to harvest algae, (2) continuous monitoring of pH and feedback control of base addition system maintains tight control of the system and allows stable operation at the target operating pH determined for the specific algae pond or bioreactor system, (3) continuous turbidity monitoring of inlet algae feed solution and aqueous effluent provide real-time performance data, (iv) no “harmful” solvents or polymers are used, (v) the biomass that is produced is not contaminated by heavy metals or solvents and can be used for feeding livestock, etc., (vi) the water/growing media from the process can be returned to the pond to be reused, and (vii) the algae remains in a wet status, which prevents costly drying and permits recycle of the water. The present inventors have tested aqueous streams having concentrations≦1.5 g/L, however, it will be understood by the skilled artisan that the process described herein can be applied to aqueous streams with higher concentrations. Doses in the range tested were not dependent on the logarithm of the cell density.

It is contemplated that any embodiment discussed in this specification can be implemented with respect to any method, kit, reagent or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 2011/0081706: Method and System for Efficient Harvesting of Microalgae and Cyanobacteria.

U.S. Patent Application Publication No. 2010/0144017: System for Harvesting Algae in Continuous Fermentation.

U.S. Pat. No. 6,524,486: Microalgae Separator Apparatus and Method.

What is claimed is:

1. A method for harvesting or separating one or more biological cells from an aqueous feed or stream comprising the steps of:
providing the aqueous feed or stream comprising the one or more biological cells in a tank or a vessel;
precipitating one or more solids in the aqueous feed or stream, wherein the one or more biological cells are associated with the precipitated solids;
allowing the precipitated solids to settle to a bottom portion of the tank or the vessel;
separating an aqueous supernatant from the settled solid precipitate; and
acidifying the settled solid precipitate, wherein the acidification results in a separation or a release of the one or more biological cells from the solid precipitate to form a concentrated slurry comprising the one or more biological cells.

2. The method of claim 1, wherein a pH of the aqueous feed or stream may be modified prior to the precipitation of the one or more solids.

3. The method of claim 2, wherein the pH of the aqueous feed or stream is modified by the addition of one or more bases, chemicals, or metals selected from the group consisting of lime, NaOH, KOH, NH₄OH, Ca(OH)₂, Mg(OH)₂, alum, aluminium chlorohydrate, aluminium sulfate, calcium oxide, iron(II) sulfate, iron(III) chloride, polyacrylamide, polyDADMAC, sodium aluminate, sodium silicate, chitosan, guar gum, alginates, and gelatin.

4. The method of claim 1, wherein the method is operated as an independent standalone operation or is incorporated or is in communication with an algal processing platform or unit.

5. The method of claim 1, wherein the method further comprises the optional steps of:
adjusting the pH of the separated aqueous supernatant, wherein the pH adjusted supernatant is discharged as an effluent or is recycled to a grow one or more biological cells; and
recirculating a portion of the flocculated solids back into the feed stream to promote faster flocculation in the feed stream
processing the concentrated slurry comprising the one or more biological cells in a processing unit to yield an oil or biodiesel, a biofuel, a pharmaceutical product, a nutraceutical product, a lipid product, or any combinations thereof, wherein the processing unit comprises:
one or more lysing units to electromechanically lyse the one or more biological cells by an application of an electromagnetic field, wherein the lysis results in a release of one or more cellular components comprising oils, neutral lipids, proteins, triglycerides, sugars or combinations and modifications thereof from the biological cells;
one or more separations unit to separate the released oils and lipids from the medium resulting in a generation of a residual biomass; and
one or more optional pumping equipment, heat exchangers, distilling equipment, reboilers, condensors, and combinations and modifications thereof.

6. The method of claim 5, wherein processing unit may optionally comprise:
one or more conversion units to convert the oils, neutral lipids or triglycerides to a fatty acid methyl ester (FAME), a biodiesel, a biofuel, a pharmaceutical product, a nutraceutical product, a lipid product, or any combinations thereof;
one or more processing units to process the residual biomass for disposal, for conversion to methane or other chemicals, for conversion to animal feed, or any combinations thereof; and
one or more storage tanks, vessels or containers to store released and separated cellular components or the processed biodiesel or biofuel.

7. The method of claim 5, wherein the step of processing the concentrated slurry comprising the one or more biological cells in a processing unit to yield an oil or biodiesel, a biofuel, a pharmaceutical product, a nutraceutical product, a lipid product, or any combinations thereof comprises the steps of:
pumping or transferring the concentrated aqueous slurry comprising the one or more biological cells to the one or more lysing units;
lysing the one or more cells electromechanically to release one or more cellular components comprising oils, neutral lipids, proteins, triglycerides, sugars or combinations and modifications thereof from the biological cells; and
separating the released oils and lipids from the medium in the separations units resulting in a generation of a residual biomass.

8. The method of claim 5, further comprising the steps of:
converting the oils, neutral lipids or triglycerides to a fatty acid methyl ester (FAME), a biodiesel or a biofuel, a pharmaceutical product, a nutraceutical product, a lipid product, or any combinations thereof; and
processing the residual biomass for disposal, for conversion to methane or other chemicals, for conversion to animal feed, or any combinations thereof.

9. The method of claim 5, wherein the processing unit may be a stationary processing plant or a modular mobile unit on a transportable platform.

10. The method of claim 9, wherein the platform is a trailer bed or a trailer.

11. The method of claim 9, wherein the platform comprises one or more sets of wheels to enable fastening onto a transportation unit.

12. The method of claim 9, wherein the modular mobile unit comprises:
one or more power supply units to provide electricity to run the lysing and separations units and to remotely operate the unit;
one or more control panels to operate and monitor the performance of the lysing and separations units.

13. The method of claim 1, wherein the one or more biological cells comprise algal cells, bacterial cells, viral cells or combinations thereof.

14. The method of claim 13, wherein the one or more algal cells comprise microalgae selected from a class comprising Bacillariophyceae, Eustigmatophyceae, and Chrysophyceae.

15. The method of claim 13, wherein the microalgal genera are selected from the group consisting of Nannochloropsis, Chlorella, Dunaliella, Scenedesmus, Selenastrum, Oscillatoria, Phormidium, Spirulina, Amphora, and Ochromonas.

16. The method of claim 13, wherein the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

17. The method of claim 1, wherein the aqueous feed or stream comprises saltwater, brackish water, fresh water, treated wastewater or combinations thereof.

18. The method of claim 1, wherein the acidification of the solid precipitate is achieved by addition of one or more acids or CO₂.

19. The method of claim 1, wherein one or more biological cells are obtained from an aqueous feed or stream harvested.

20. A method for harvesting or separating one or more microalgal cells from an aqueous feed or stream comprising the steps of:
providing the aqueous feed or stream comprising the one or more microalgal cells in a tank or a vessel, wherein the aqueous feed or stream comprises saltwater, brackish water, fresh water, treated wastewater or combinations thereof;
raising a pH of the aqueous feed or stream by an addition of a base;
precipitating one or more solids in the aqueous feed or stream, wherein the one or more microalgal cells are associated with the precipitated solids;
allowing the precipitated solids to settle to a bottom portion of the tank or the vessel;
separating an aqueous supernatant from the settled solid precipitate; and
contacting the settled solid precipitate with CO₂ or other acid to acidify the solid precipitate, wherein the acidification results in a separation or a release of the one or more microalgal cells from the solid precipitate to form a concentrated slurry comprising the one or more microalgal cells.

21. The method of claim 20, wherein the method further comprises the optional steps of:
adjusting the pH of the separated aqueous supernatant, wherein the pH adjusted supernatant is discharged as an effluent or is recycled to a grow one or more microalgal cells; and
processing the concentrated slurry comprising the one or more microalgal cells in a processing unit to yield an oil or biodiesel in a system comprising:
one or more lysing units to electromechanically lyse the one or more microalgal cells by an application of an electromagnetic field, wherein the lysis results in a release of one or more cellular components comprising oils, neutral lipids, proteins, triglycerides, sugars or combinations and modifications thereof from the microalgal cells;
one or more separations unit to separate the released oils and lipids from the medium resulting in a generation of a residual biomass; and
one or more optional pumping equipment, heat exchangers, distilling equipment, reboilers, condensors, and combinations and modifications thereof.

22. The method of claim 21, wherein the step of processing the concentrated slurry comprising the one or more biological cells in a processing unit to yield an oil or biodiesel comprises the steps of:
pumping or transferring the concentrated aqueous slurry comprising the one or more microalgal cells to the one or more lysing units;
lysing the one or more cells electromechanically to release one or more cellular components comprising oils, neutral lipids, proteins, triglycerides, sugars or combinations and modifications thereof from the microalgal cells; and
separating the released oils and lipids from the medium in the separations units resulting in a generation of a residual biomass.

23. The method of claim 22, further comprising the steps of:
converting the oils, neutral lipids or triglycerides to a fatty acid methyl ester (FAME), a biodiesel or a biofuel, a pharmaceutical product, a nutraceutical product, a lipid product, or any combinations thereof; and
processing the residual biomass for disposal, for conversion to methane or other chemicals, for conversion to animal feed, or any combinations thereof.

24. The method of claim 21, wherein processing unit may optionally comprise:
one or more conversion units to convert the oils, neutral lipids or triglycerides to a fatty acid methyl ester (FAME), a biodiesel or a biofuel, a pharmaceutical product, a nutraceutical product, a lipid product, or any combinations thereof;
one or more processing units to process the residual biomass for disposal or for conversion to methane or other chemicals; and
one or more storage tanks, vessels or containers to store released and separated cellular components or the processed biodiesel or biofuel.

25. The method of claim 24, wherein the processing unit may be a modular mobile unit on a transportable platform, wherein the platform is a trailer bed or a trailer and comprises one or more sets of wheels to enable fastening onto a transportation unit.

26. The method of claim 25, wherein the modular mobile unit comprises:
one or more power supply units to provide electricity to run the lysing and separations units and to remotely operate the unit;
one or more control panels to operate and monitor the performance of the lysing and separations units.

27. The method of claim 20, wherein the one or more microalgal cells comprise microalgae selected from a class comprising Bacillariophyceae, Eustigmatophyceae, and Chrysophyceae.

28. The method of claim 20, wherein a microalgal genera are selected from the group consisting of Nannochloropsis, Chlorella, Dunaliella, Scenedesmus, Selenastrum, Oscillatoria, Phormidium, Spirulina, Amphora, and Ochromonas.

29. The method of claim 20, wherein a microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

30. One or more microalgal cells harvested by the method of claim 20.

31. An apparatus for producing a biodiesel, a fatty acid methyl ester (FAME), a biofuel or combinations and modifications thereof from a microalgal cell culture comprising:
an algal growth tank or a cultivation tank comprising an aqueous feed or stream for growing the one or more algal species in presence of water and other growth factors selected from the group consisting of nutrients, minerals, CO₂, air, and light;
a harvesting tank for separating or harvesting the microalgal cell culture from the aqueous feed or stream, wherein the aqueous feed or stream comprises saltwater, brackish water, fresh water, treated wastewater or combinations thereof, wherein the method of harvesting or separating the microalgal cell culture comprises the steps of:
raising a pH of the aqueous feed or stream by an addition of a base;
precipitating one or more solids in the aqueous feed or stream, wherein the microalgal cells are associated with the precipitated solids;
allowing the precipitated solids to settle to a bottom portion of the tank or the vessel;
separating an aqueous supernatant from the settled solid precipitate; and
contacting the settled solid precipitate with CO₂ or other acid to acidify the solid precipitate, wherein the acidification results in a separation or a release of the microalgal cells from the solid precipitate to form a concentrated slurry comprising the one or more microalgal cells; and
a processing unit for processing the concentrated slurry of the microalgal cells comprising:
one or more lysing units to electromechanically lyse the one or more microalgal cell culture by an application of an electromagnetic field, wherein the lysis results in a release of one or more cellular components comprising oils, neutral lipids, proteins, triglycerides, sugars or combinations and modifications thereof from the algal cells;
one or more separations unit to separate the released oils and lipids from the medium resulting in a generation of a residual biomass;
a reaction vessel for converting the separated algal lipids, triglycerides to a biodiesel, a FAME, a biofuel or combinations or modifications thereof by a transesterification reaction; and
one or more optional pumping equipment, heat exchangers, distilling equipment, reboilers, condensors, and combinations and modifications thereof.
one or more power supply units to provide electricity to run the dewatering, lysing, and separations units and to remotely operate the unit;
one or more control panels to operate and monitor the performance of the dewatering, lysing, and separations units;

32. The apparatus of claim 31, wherein the processing unit may be a modular mobile unit on a transportable platform, wherein the platform is a trailer bed or a trailer and comprises one or more sets of wheels to enable fastening onto a transportation unit.

33. The apparatus of claim 32, wherein the modular mobile unit comprises:
one or more power supply units to provide electricity to run the lysing and separations units and to remotely operate the unit;
one or more control panels to operate and monitor the performance of the lysing and separations units.

34. The apparatus of claim 32, wherein the modular mobile unit can be manually or electronically operated by an onsite operator, wherein electronic operation is achieved by use of one or more sensors, wireless or wired control systems.

35. The apparatus of claim 32, wherein the modular mobile unit can be remotely operated.

36. The apparatus of claim 31, wherein the apparatus comprises an optional processing unit for processing the residual biomass for disposal, for conversion to methane or other chemicals, for conversion to animal feed, or any combinations thereof.

37. The apparatus of claim 31, wherein the concentrated microalgal cell culture may or may not be concentrated prior to lysing and oil separation, wherein the concentration is done by a centrifugation or any other suitable concentration process.

38. The apparatus of claim 31, wherein the microalgal cell culture is selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, Pleurochysis, and Labyrinthuila sp.

39. The apparatus of claim 31, wherein a microalgal genera are selected from the group consisting of Nannochloropsis, Chlorella, Dunaliella, Scenedesmus, Selenastrum, Oscillatoria, Phormidium, Spirulina, Amphora, and Ochromonas.

40. The apparatus of claim 31, wherein a microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

41. The apparatus of claim 31, wherein the apparatus is capable of operation in a batch or a continuous processing mode.

42. The apparatus of claim 31, wherein the one or more lysing units comprise electromechanical lysing units, sonicators, ultrasound devices, pressure homogenizers, high speed homogenizers, osmotic shock inducing devices or devices for chemical or enzymatic lysis.

43. The apparatus of claim 31, wherein the separation units comprise non-dispersive separation devices, decantation units, liquid-liquid extraction units, solvent assisted extraction units or combinations and modifications thereof.

44. The apparatus of claim 31, wherein the microalgal culture is Chlorella or Nannochloropsis.
